(19) United States
(12) Patent Application Publication
(10) Pub. No.: US 2007/0086974 A1
(43) Pub. Date: Apr. 19, 2007

(54) CETIRIZINE COMPOSITIONS

(76) Inventors: Rahul Sudhakar Gawande, Nagpur 440 015, Maharashtra (IN); Kodipyaka Ravinder, Kaghaznagar 504 296, A.P. (IN); Praveen Kumar B.S., Coorg 571 235, Karnataka (IN); Indu Bhushan, Hyderabad 500 072, A.P. (IN); Mailatur Sivaraman Mohan, Hyderabad 500 072, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/539,406

(22) Filed: Oct. 6, 2006

Related U.S. Application Data
(60) Provisional application No. 60/745,249, filed on Apr. 20, 2006.

(30) Foreign Application Priority Data
Oct. 6, 2005 (IN) 1418/CHE/2005

(51) Int. Cl. A61K 31/787

(52) U.S. Cl. 424/78.15; 521/32

(57) ABSTRACT

Stable and palatable taste masked pharmaceutical compositions of substituted benzhydrylpiperazines and processes for preparing them.

### INTRODUCTION TO THE INVENTION

The present invention relates to pharmaceutical compositions of substituted benzhydrylpiperazines or their pharmaceutically acceptable salts, solvates, polymorphs, enantiomers or mixtures; processes for preparing the same; and their methods of use.

Further, the present invention also relates to stable and palatable taste masked pharmaceutical compositions of substituted benzhydrylpiperazines in combination with a resin and the processes for preparing the same.

Benzhydrylpiperazines are a class of drugs useful as antiallergens, spasmolytics and antihistaminics that are generally non-sedative.

Antihistamines (H₁ receptor antagonists) act by competitively antagonizing the effects of histamine at receptor sites. They do not block the release of histamine and hence offer only palliative relief of the allergic symptoms. They are effective in mild, local allergic and minor drug and serum reactions characterized by pruritis.

Cetirizine chemically is (±)-[2[4-[(4-chlorophenyl)phenylmethyl]-1-piperazinyl]ethoxy]acetic acid or [2-[4-(p-chloro-α-phenylbenzyl)-1-piperazinyl]ethoxy]acetic acid, with the structural Formula I. It is used in commercial products as a dihydrochloride salt. It is a piperazine derivative and a metabolite of hydroxyzine. It is an orally active, selective H₁-receptor antagonist and useful as a non-sedating antihistamine. Cetirizine dihydrochloride is bitter in taste and is reported to be long acting with some mast-cell stabilizing activity. It is used for the symptomatic relief of allergic conditions including rhinitis and chronic urticaria. Cetirizine is prescribed for the treatment of seasonal allergies in patients aged 2 years and older.

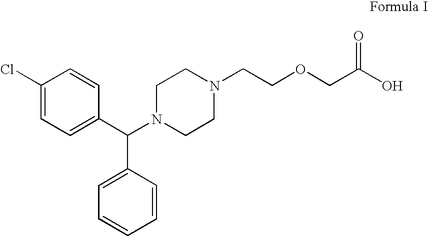

The commercially available products of cetirizine are film-coated, immediate release oral tablets and chewable tablets, both containing 5 mg or 10 mg of cetirizine dihydrochloride under the brand name ZYRTEC® and a sweet flavored ZYRTEC® syrup containing cetirizine dihydrochloride at a concentration of 1 mg/ml for pediatric use, all sold by Pfizer.

Contact of cetirizine should be avoided with alcohols having a molecular weight less than 100 as it results in a reaction with cetirizine, usually esterification, and thereby leads to instability of the dosage form.

Taste has different components like sweet, sour, saline, bitter, acidic, alkaline, bland, astringent and metallic. Taste masking of each type will differ with respect to the taste that is to be masked, substances that give the relatively disagreeable taste, substances used for masking the taste and the process of taste masking. Hence the art of taste masking is relatively complex and involves many variables and factors, which are case specific.

For active ingredients, which have a disagreeable bitter taste, unpalatability of a medication is a major problem. Moreover, this problem is not limited to liquid oral compositions like solutions, syrups and suspensions but also for chewable or dispersible tablets where the solid or liquid oral dosage forms usually lead to perceptible exposure of actives to the taste buds.

The conventional methods for taste masking, like coating of drugs by polymeric materials such as water-soluble or -insoluble celluloses or lipids or sugar and the use of sweeteners such as sugar, sorbitol, sodium saccharin in the composition, do not provide complete taste masking over a period of time. The taste masking by water-soluble sugar coating still gives the bitter taste once the sugar coat gets dissolved in the mouth. The use of water-insoluble coatings frequently affects the release profile of the drug, thereby affecting its bioavailability.

U.S. Pat. No. 3,558,600 describes a method for masking the bitter taste of antihistamines belonging to the family of substituted 1-(p-chlorobenzhydryl) piperazine.

U.S. Pat. No. 6,455,533 and International Application Publication No. WO 99/01133 disclose a method for masking the taste of active ingredients by forming an inclusion complex with a cyclodextrin.

U.S. Patent Application Publication No. 2006/0115529 and International Application Publication No. WO 2006/061700 describe resinates of cetirizine or its pharmaceutically acceptable salts and fast disintegrating compositions thereof.

A. H. Kibbe, Ed., Handbook of Pharmaceutical Excipients (Third Edition), American Pharmaceutical Association, Washington, D.C., 2000, discloses the use of polacrilin ion-exchange resins as excipients to stabilize, mask or modify the taste of drugs. It also discloses the use of polacrilin ion-exchange resins in the preparation of sustained-release dosage forms of antihistaminics. Also, the product data sheets for AMBERLITE™ IRP (polacrilin potassium NF) resins from Rohm and Haas Co. describe uses for taste masking, stabilization of vitamins and sustained release applications.

A pharmaceutical composition which masks the taste of active ingredients having a disagreeable bitter taste without affecting its efficacy and leading to the stabilization of the formulation would be a significant improvement in the field of taste masked pharmaceutical compositions.

These and other needs are addressed by this invention.

### SUMMARY OF THE INVENTION

The present invention provides stable and palatable taste masked pharmaceutical compositions of substituted benzhydrylpiperazines or their pharmaceutically acceptable salts, solvates, enantiomers or mixtures and processes for preparing the same.

An embodiment of the invention includes a complex formed from cetirizine, or a salt thereof, and an ion exchange resin, wherein a weight ratio of cetirizine or salt to resin is about 1:3 to about 1:5.

Another embodiment of the invention includes a process for preparing a pharmaceutical tablet, comprising forming granules from ion exchange resin particles and an aqueous solution of cetirizine or a salt thereof, and combining granules with at least one pharmaceutical excipient.

A further embodiment of the invention comprises a pharmaceutical tablet composition containing:

about 10 to about 25 weight percent of ion exchange resin particles comprising cetirizine, wherein a weight ratio of cetirizine to resin is about 1:3 to about 1:5;

about 40 to about 70 percent by weight mannitol;

about 3 to about 7 weight percent copovidone;

about 5 to about 10 weight percent microcrystalline cellulose; and

about 2 to about 5 weight percent of a lubricant.

### DETAILED DESCRIPTION OF THE INVENTION

The present invention relates to stable and palatable taste masked pharmaceutical compositions of substituted benzhydrylpiperazines or their pharmaceutically acceptable salts, solvates, enantiomers or mixtures and processes for preparing the same.

In one embodiment of the present invention, substituted benzhydrylpiperazines have been found to be useful such as cetirizine, efletirizine, buclizine, etodroxizine, hydroxyzine, chlorcyclizine and the like.

In another embodiment, cetirizine or its pharmaceutically acceptable salts have been found to be useful in the present invention.

In yet another embodiment, the present invention provides a dose of cetirizine from about 5 to about 10 mg per unit.

Cetirizine, under normal conditions of processing and storage is known to be reactive toward low molecular weight monohydric and polyhydric alcohols, which are conventionally used as solvents (such as methanol, ethanol, isopropanol and glycerin) or as plasticizers or as other pharmaceutical aids (such as mannitol, xylitol, sorbitol, dextrose, sucrose) in the formulation.

Cetirizine in the presence of certain polyols such as xylitol, mannitol, sorbitol, dextrose, sucrose, maltodextrins and polysaccharides and the like results in undesired reaction usually esterifications with undesired reaction products, thereby damaging the dosage form.

Also, not all components containing the alcohol group are reactive towards cetirizine under normal conditions of processing and storage (temperatures less than 100° C.). Examples include cellulosic materials containing free hydroxyl groups such as microcrystalline celluloses, cellulose ethers and esters.

In another embodiment, cetirizine is a bitter tasting drug and formulating chewable tablets of cetirizine poses challenge to the formulator, as it interacts with normally used chewable formulation excipients such as xylitol, mannitol and other polyol excipients as mentioned above leading to an unstabalised product.

The present invention includes the use of an ion exchange resin to formulate a stable and palatable dosage form of cetirizine.

The ion exchange resin used is either a cation exchange resin or an anion exchange resin. Ion exchange resins useful in the practice of the present invention include but are not limited to anionic resins such as DUOLITE™ AP143/1083 (cholestyramine resin USP), and cationic resins such as AMBERLITE™ IRP64 (a porous copolymer of methacrylic acid crosslinked with divinylbenzene). DUOLITE and AMBERLITE resins are available from Rohm and Haas Co., Philadelphia, Pa. U.S.A.

In one embodiment, AMBERLITE IRP64, which is an insoluble, weakly acidic, hydrogen form, cation exchange resin is used as an ion exchange resin to form a stable resinate.

The w/w ratios of the amount of ion exchange resin to the amount of cetirizine to form a stable and palatable resinate complex are about 5:1 to 1:5 or about 3:1 to 1:3, respectively. In specific embodiments, a weight ratio of cetirizine or a salt thereof to resin is about 1:3 to about 1:5.

In a specific embodiment, a resinate is prepared by dispersing three parts of polacrilin resin (e.g., AMBERLITE IRP64) in water under high speed stirring for about 2 hours. One part of cetirizine dihydrochloride is added to the above mentioned dispersion and the stirring is continued for about 3 hours. The dispersion so obtained is filtered and the resinate obtained is dried at 60° C. until loss on drying (LOD at 105° C.) is less than about 10% w/w. The dried cetirizine polacrilin resinate is then sifted through an ASTM # 40 mesh sieve before use.

In one embodiment, the present invention provides stable and palatable pharmaceutical compositions comprising cetirizine or pharmaceutically acceptable salts, solvates, enantiomers or mixtures thereof, blended or granulated with an ion exchange resin and other pharmaceutically acceptable excipients to form a resinate.

Any pharmaceutically acceptable excipient known in the art can be used in a chewable tablet formulation to provide adequate compression and to make up the tablet mass, resulting in a dosage form that is easier for the patient and caregiver to handle.

Common diluents that can be used in pharmaceutical formulations include microcrystalline cellulose (MCC), silicified MCC (e.g., PROSOLV™ HD 90), microfine cellulose, lactose, starch, pregelatinized starch, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide and the like.

Binders can be included in the pharmaceutical compositions of the present invention to help hold a tablet together after compression. Some typical binders are acacia, guar gum, alginic acid, carbomer (e.g., CARBOPOL®), dextrin, maltodextrin, methylcellulose, ethyl cellulose, hydroxyethyl cellulose, hydroxypropyl cellulose (e.g. KLUCEL®), hydroxypropyl methylcellulose (e.g., METHOCEL®), carboxymethylcellulose sodium, liquid glucose, magnesium aluminum silicate, polymethacrylates, crospovidones (e.g., POLYPLASDONE®), povidones (e.g., Povidone K90 D, KOLLIDON®, PLASDONE®), copovidones (copolymers of 1-vinyl-2-pyrrolidone and vinyl acetate), gelatin, starch and mixtures thereof.

The pharmaceutical compositions of the present invention can be made into tablets, particularly chewable, dispersible, or mouth dissolving tablets, include sweeteners such as, but not limited to: natural sweeteners such as sucrose, dextrose, fructose, invert sugar, mannitol, sorbitol and the like; and synthetic sweeteners such as saccharin, aspartame, acesulfame potassium, cyclamates and the like. The amount of sweetener may vary depending on the sweetening strength of the particular sweetener used. Mixtures of any two or more sweeteners are useful in the invention.

Pharmaceutical compositions of the present invention can further include glidants (e.g., talc, colloidal silicon dioxide, magnesium trisilicate, powdered cellulose, starch, tribasic calcium phosphate and the like), lubricants (e.g., stearates such as magnesium stearate, magnesium stearate, calcium stearate and zinc stearate; corn starch talc and the like), flavoring agents, colorants, and other commonly used excipients.

In one embodiment, the pharmaceutical compositions comprising cetirizine-resinate is made into tablets, caplets, capsules (hard or soft gelatin chewable capsules) and the like.

The present invention provides a solution for a long felt need for the stabilization of compositions of cetirizine by formulating it in combination with a resin thus preventing the undesirable reaction of cetirizine with hydroxy and other groups of any reactive tablet excipient components, such as diluents, thereby avoiding the decomposition of drug in the final dosage form and also rendering a stable composition with no significant impurities, even when subjected to stress storage conditions of about 40° C. or about 50° C. at a relative humidity of about 75%.

The following examples will further illustrate certain specific aspects and embodiments of the invention in greater detail and are not intended to limit the scope of the invention.

### EXAMPLE 1

### Cetirizine Polacrilin Resinate

1. 300 g of polacrilin resin (Amberlite IRP64®) was dispersed in 2000 g of water under high speed stirring for 2 hours.

2. 100 g of cetirizine dihydrochloride was added to the dispersion of step 1 and the stirring was continued for a further 3 hours.

3. The dispersion of step 2 was filtered and was dried at 60° C. in a tray drier to get a loss on drying (LOD) below 10% w/w (as measured at 105° C.).

4. The dried cetirizine polacrilin resinate of step 3 was sifted through an ASTM # 40 mesh sieve.

5. The dried and sifted cetirizine polacrilin resinate was analyzed for drug content.

The drug content in cetirizine polacrilin resinate was found to be about 18% w/w.

### EXAMPLE 2

### Cetirizine Polacrilin Resinate

1. 150 g of cetirizine dihydrochloride was dissolved in 900 mL of purified water.

2. Amberlite IRP 64 (600 g) was taken in a beaker and granulated with cetirizine dihydrochloride solution of step 1 to form a wet granular mass.

3. The wet granular mass of step 2 was dried in a tray drier at 60° C. until the LOD (at 105° C.) was 5.25% w/w.

4. The dried resinate thus formed was sifted through an ASTM # 40 mesh sieve.

The drug content in cetirizine polacrilin resinate was found to be about 19% w/w.

### EXAMPLE 3

### Composition of Cetirizine 10 mg Chewable Tablet (with Resinate)

| Ingredients | Quantity/Batch (g) |
|---|---|
| Cetirizine polacrilin resinate of Example 1 | 27.8 |
| Mannitol (Pearlitol SD 200) # | 139.3 |
| Crospovidone ## | 6 |
| Microcrystalline cellulose (Avicel PH 102) $ | 15 |
| Acesulfame potassium $$ | 7.5 |
| Colloidal silicon dioxide | 1 |
| Cooling flavor * | 0.2 |
| Peppermint flavor ** | 1 |
| Magnesium stearate | 2.3 |

# Roquette America Inc. manufactures Pearlitol SD 200.
## Crospovidone is a synthetic, insoluble but rapidly swellable, crosslinked homopolymer of N-vinyl-2-pyrrolidone manufactured by BASF.
$ FMC BioPolymer manufactures Avicel PH 102.
$$ Sunett ™, Frankfort, Germany, manufactures acesulfame potassium.
* Cooling flavor is S-124827 (Permaseal ®) manufactured by Givaudan, USA
** Peppermint flavor is 76175-51 (Permaseal ®) manufactured by Givaudan, USA

Manufacturing Process:

1. Cetirizine polacrilin resinate of Example 1, crospovidone, microcrystalline cellulose, cooling flavor, peppermint flavor, and acesulfame potassium were sifted through an ASTM # 60 mesh sieve.

2. Mannitol and colloidal silicon dioxide were sifted through an ASTM # 40 mesh sieve.

3. The sifted ingredients of steps 1 and 2 were blended together in a double cone blender for 20 minutes.

4. Talc and magnesium stearate were sifted together through an ASTM # 60 mesh sieve, added to the double cone blender, and blended for 5 minutes.

5. The lubricated blend of step 4 was compressed into tablets using 10 mm round punches in rotary compression machine

Tablet Parameters:

| | | |
|---|---|---|
| Average weight | 404 | mg |
| Hardness | 4-6 | kp |
| Disintegration time | 20-35 | sec |
| Friability | 0.102% | |
| Taste evaluation* | 1-2 | |

*Rating from 0 to 5: 5 being highly bitter, 0 being no bitterness.

In Vitro Dissolution Study:

The in vitro dissolution profile of tablets of Example 3 was compared with ZYRTEC® 10 mg (cetirizine hydrochloride) chewable tablets.

Media: 0.1 N hydrochloric acid

Apparatus: USP type 2 [“Apparatus 2” in Test 711—Dissolution, United States Pharmacopeia 24, United States Pharmacopeial Convention, Inc., Rockville, Md. U.S.A., page 1942 (2000)].

Stirring speed: 50 rpm

Volume: 500 mL

Temperature: 37.5±0.5° C.

% Drug Released

| Time | | ZYRTEC ® 10 mg |
|---|---|---|
| (minutes) | Example 3 | Chewable Tablets |
| 10 | 77 | 80 |
| 20 | 88 | 91 |

### EXAMPLE 4 (Comparative)

### Composition of Cetirizine 10 mg Chewable Tablet (without Resinate)

| | Quantity/Batch |
|---|---|
| Ingredients | (g) |
| Cetirizine dihydrochloride | 15 |
| Mannitol (Pearlitol SD 200) | 462.8 |
| Crospovidone | 24 |
| Microcrystalline cellulose (Avicel PH 102) | 45 |
| Acesulfame potassium | 27 |
| Colloidal silicon dioxide | 3 |
| Cooling flavor | 0.8 |
| Peppermint flavor | 3 |
| Talc | 12 |
| Magnesium stearate | 7.5 |

Manufacturing Process:

1. Cetirizine dihydrochloride, crospovidone, microcrystalline cellulose, cooling flavor, peppermint flavor, and acesulfame potassium were sifted through an ASTM # 60 mesh sieve.

2. Mannitol and colloidal silicon dioxide were sifted through an ASTM # 40 mesh sieve.

3. The sifted ingredients of step 1 and 2 were blended together in a double cone blender for 20 minutes.

4. Talc and magnesium stearate were sifted together through a 60 ASTM mesh sieve, added to the double cone blender, and blended for 5 minutes.

5. The lubricated blend of step 4 was compressed into tablets using 9 mm round standard concave punches in a rotary compression machine

Tablet Parameters:

| | | |
|---|---|---|
| Average weight | 401 | mg |
| Hardness | 6-7 | kp |
| Disintegration time | 18-25 | seconds |
| Friability | 0.58% | |
| Taste evaluation* | 4-5 | |

*Rating from 0 to 5: 5 being highly bitter, 0 being no bitterness

Since a resinate complex was not used to mask the bitterness of the drug compound, the taste evaluation ratings were higher than for the Example 3 tablets.

### EXAMPLES 5 and 6

### Compositions of Cetirizine Chewable Tablets

| | Example 5 | Example 6 |
|---|---|---|
| Ingredients | mg/Tablet | mg/Tablet |
| Cetirizine polacrilin | 55.2 (equivalent | 55.2 (equivalent |
| resinate of Example 1 | to 10 mg of | to 10 mg of |
| | cetirizine HCl) | cetirizine HCl) |
| Mannitol (Pearlitol SD 200) | 287.8 | 170.3 |
| Crospovidone | 12 | — |
| Microcrystalline cellulose | 30 | 20 |
| (Avicel PH 102) | | |
| Colloidal silicon dioxide | 2 | 2 |
| Talc | 8 | — |
| Magnesium stearate | 5 | 2.5 |
| Tablet weight | 400 mg | 250 mg |

Manufacturing Process:

1. Cetirizine polacrilin resinate of Example 1, crospovidone (if included), and microcrystalline cellulose were sifted through an ASTM # 60 mesh sieve.

2. Mannitol and colloidal silicon dioxide were sifted through an ASTM # 40 mesh sieve.

3. The sifted ingredients of steps 1 and 2 were blended together in a double cone blender for 20 minutes.

4. Talc (if included) and magnesium stearate were sifted together through an ASTM # 60 mesh sieve, added to the double cone blender, and blended for 5 minutes.

5. The lubricated blend of step 4 was compressed into tablets.

### EXAMPLE 7

### Composition of Cetirizine 10 mg Chewable Tablet

| | Quantity/Batch |
|---|---|
| Ingredients | (g) |
| Cetirizine polacrilin resinate of Example 2 | 315.6 |
| Mannitol (Pearlitol SD 200) | 1493.4 |
| Crospovidone | 96 |
| Copovidone (Plasdone S-630)*** | 120 |
| Microcrystalline cellulose (Avicel PH 102) | 180 |
| Acesulfame potassium | 90 |
| Colloidal silicon dioxide | 12 |
| Tuttifrutti flavor* | 12 |
| Frescofort flavor** | 3 |
| Talc | 48 |
| Magnesium stearate | 30 |

*Tuttifrutti flavor manufactured by Givaudan, USA
**Frescofort flavor manufactured by Givaudan, USA
***Plasdone ™ S-630 supplied by International Specialty Products, USA is a 2:3 by weight copolymer of vinyl acetate and 1-vinyl-2-pyrrolidone, having a K value between 25.2 and 30.8.

Manufacturing Process:

1. Cetirizine polacrilin resinate of Example 2, crospovidone, microcrystalline cellulose, Plasdone S-630, flavors, and acesulfame potassium were sifted through an ASTM # 60 mesh sieve.

2. Mannitol and colloidal silicon dioxide were sifted through an ASTM # 40 mesh sieve.

3. The sifted ingredients of steps 1 and 2 were blended together in a double cone blender for 20 minutes.

4. Talc and magnesium stearate were sifted together through an ASTM # 60 mesh sieve and added to the double cone blender and blended for 5 minutes.

5. The lubricated blend of step 4 was compressed into tablets using 10.5 mm round punches in a 21 station single rotary compression machine.

Tablet Parameters

| | | |
|---|---|---|
| Average weight | 401 | mg |
| Hardness | 6-7 | kp |
| Disintegration time | 40-50 | seconds |
| Friability | 0.18% | |
| Taste evaluation* | 1-2 | |

*Rating from 0 to 5: 5 being highly bitter, 0 being no bitterness.

### EXAMPLE 8

### Compositions of Cetirizine Chewable Tablet 5 mg and 10 mg

A. Composition of Cetirizine dihydrochloride-polacrilex resin granules.

| Ingredients | Quantity/Batch (Kg) |
|---|---|
| Cetirizine dihydrochloride | 7.57 |
| Polacrilex resin (Amberlite IRP64) | 30 |
| Sodium hydroxide | 1.5 |
| Water | 30.7 |

Drug content of Cetirizine dihydrochloride-polacrilex resin granules: 19.1% w/w.

B. Composition of Cetirizine hydrochloride chewable tablets 5 mg and 10 mg.

| Ingredients | Quantity/Batch (Kg) |
|---|---|
| Cetirizine-resin granules of A | 15.7 |
| Mannitol | 72.9 |
| Acesulfame potassium | 5.4 |
| Crospovidone | 4.8 |
| Copovidone (Plasdone S 630) | 6 |
| Peppermint flavor | 0.9 |
| Cooling flavor | 0.5 |
| Microcrystalline cellulose (Avicel PH102) | 9 |
| Colloidal silicon dioxide | 0.6 |
| Talc | 2.4 |
| Magnesium stearate | 1.8 |

Manufacturing Process:

1. Cetirizine dihydrochloride was dissolved in an aqueous solution of the sodium hydroxide.

2. pH of the solution of step 1 was adjusted to 6±0.5 with sodium hydroxide solution.

3. Polacrilex resin was loaded into a rapid mixer granulator (RMG) and was granulated with the solution of step 2.

4. Granules were dried in a tray drier at about 60° C. until loss on drying was about 5% w/w.

5. Dried granules were sifted through an ASTM 60 mesh sieve.

6. Granules of step 5 were blended with other excipients, except talc and magnesium stearate.

7. Blend of step 6 was lubricated using talc and magnesium stearate.

8. Lubricated blend of step 7 was compressed into tablets in a rotary compression machine using 8.1 mm round punch set to an average tablet weight of 200 mg for the 5 mg cetirizine tablets, and using a 10.6 mm round punch set to an average tablet weight of 400 mg for the 10 mg cetirizine tablets.

Tablet Parameters (10 mg Tablets):

| | | |
|---|---|---|
| Average weight | 402 | mg |
| Hardness | 6-7 | kp |
| Disintegration time | 40-55 | seconds |
| Friability | 0.112% | |
| Taste evaluation* | 2-3 | |

*Rating from 0 to 5; 5 being highly bitter; 0 being no bitterness

In Vitro Dissolution Study:

Media: 0.1 N hydrochloric acid

Apparatus: USP type 2 [“Apparatus 2” in Test 711—Dissolution, United States Pharmacopeia 24, United States Pharmacopeial Convention, Inc., Rockville, Md. U.S.A., page 1942 (2000)].

Stirring speed: 75 rpm

Volume: 900 mL Temperature: 37±0.5° C.

| | % Drug Released | | | |
|---|---|---|---|---|
| | Cetirizine | ZYRTEC ® | Cetirizine | ZYRTEC ® |
| Time | Tablets 5 mg | 5 mg | Tablets 10 mg | 10 mg |
| (minutes) | Example 8 | Tablets | Example 8 | Tablets |
| 0 | 0 | 0 | 0 | 0 |
| 10 | 91 | 98 | 92 | 89 |
| 20 | 93 | 99 | 94 | 95 |
| 30 | 94 | 100 | 95 | 95 |

In Vivo Study:

A randomized, two treatment, two period, two sequence, single dose, crossover bioequivalence study was conducted in healthy human subjects under fasted and fed states (n=28). The pharmacokinetic data were as follows:

| | Bioequivalence Study Results | | | |
|---|---|---|---|---|
| | Fasted State | | Fed State | |
| | Cetirizine | ZYRTEC ® | Cetirizine | ZYRTEC ® |
| | Tablets | 10 mg | Tablets | 10 mg |
| | 10 mg | Chewable | 10 mg | Chewable |
| Parameters | Example 8 | Tablets | Example 8 | Tablets |
| Cₘₐₓ (ng/ml) | 282.4 | 323.8 | 200.2 | 216.9 |
| AUC ₀₋ₜ | 3095.3 | 2976.5 | 2839 | 2780.2 |
| (ng · hr/ml) | | | | |
| AUC _{0-inf} | 3222.8 | 3099.4 | 3024.2 | 2918.3 |
| (ng · hr/ml) | | | | |

We claim:

1. A complex formed from cetirizine, or a salt thereof, and an ion exchange resin, wherein a weight ratio of cetirizine or salt to resin is about 1:3 to about 1:5.

2. The complex of claim 1, wherein an ion exchange resin is a copolymer of methacrylic acid and divinylbenzene.

3. A pharmaceutical composition containing the complex of claim 1 and at least one pharmaceutical excipient.

4. A pharmaceutical composition containing the complex of claim 2 and at least one pharmaceutical excipient.

5. A pharmaceutical tablet composition containing about 10 to about 25 weight percent of the complex of claim 1, about 40 to about 70 percent by weight mannitol, about 3 to about 7 weight percent copovidone, about 5 to about 10 weight percent microcrystalline cellulose, and about 2 to about 5 weight percent of a lubricant.

6. A process for preparing a pharmaceutical tablet, comprising forming granules from ion exchange resin particles and an aqueous solution of cetirizine or a salt thereof, and combining granules with at least one pharmaceutical excipient.

7. The process of claim 6, wherein an ion exchange resin is a copolymer of methacrylic acid and divinylbenzene.

8. The process of claim 6, wherein a weight ratio of cetirizine or salt to resin is about 1:3 to about 1:5.

9. The process of claim 6, wherein a weight ratio of cetirizine or salt to resin is about 1:3 to about 1:5.

10. The process of claim 6, wherein an aqueous solution has a pH about 6.

11. A pharmaceutical tablet composition containing:
about 10 to about 25 weight percent of ion exchange resin particles comprising cetirizine, wherein a weight ratio of cetirizine to resin is about 1:3 to about 1:5;
about 40 to about 70 percent by weight mannitol;
about 3 to about 7 weight percent copovidone;
about 5 to about 10 weight percent microcrystalline cellulose; and
about 2 to about 5 weight percent of a lubricant.

12. The pharmaceutical tablet composition of claim 11, wherein an ion exchange resin is a copolymer of methacrylic acid and divinylbenzene.

13. The pharmaceutical tablet composition of claim 11, wherein resin particles are granules, prepared by granulating ion exchange resin particles using a solution comprising cetirizine or a salt thereof.

14. The pharmaceutical tablet composition of claim 11, wherein resin particles are prepared by contacting ion exchange resin particles with a solution comprising cetirizine or a salt thereof, for a time sufficient for the resin particles to adsorb citirizine.
